(19) United States
(12) Patent Application Publication
(10) Pub. No.: US 2011/0092557 A1
(43) Pub. Date: Apr. 21, 2011

(54) Neuraminidase Inhibitors And Compositions And Methods Related Thereto

(75) Inventors: Xiaodong Fan, Center Valley, PA (US); Lidia Cristian, Princeton Jct., NJ (US)

(73) Assignee: INFLUMEDIX INC., Radnor, PA (US)

(21) Appl. No.: 12/490,650

(22) Filed: Jun. 24, 2009

Related U.S. Application Data
(60) Provisional application No. 61/075,368, filed on Jun. 25, 2008.

(51) Int. Cl. A61K 31/4172; C07C 205/56; C07C 255/59; C07D 233/64; C07D 207/16; C12N 9/99; A61K 31/198; A61K 31/401; A61P 31/04; A61P 31/16

(52) U.S. Cl. 514/398; 562/437; 558/414; 548/336.1; 548/532; 435/184; 514/567; 514/564; 514/423

(57) ABSTRACT

The present invention provides compounds comprising amino acid R groups, compositions comprising the same, and methods of inhibiting neuraminidase and/or treating influenza, Pseudomonas aeruginosa, or Bacteroides fragilis infection in a mammal.

### CROSS-REFERENCE TO RELATED APPLICATIONS

This application claims priority to U.S. provisional application Ser. No. 61/075,368 filed Jun. 25, 2008, which is incorporated herein by reference in its entirety.

### FIELD OF THE INVENTION

The present invention is directed, in part, to compounds, and compositions thereof, that inhibit neuraminidase, and to methods of using the same.

### BACKGROUND OF THE INVENTION

Influenza is one of the most serious infectious diseases worldwide and has been a major threat to public health. It is estimated that 10 to 20% of the general population are infected with influenza virus each year. Despite considerable knowledge of viral infectivity, no therapeutic measure is highly and specifically effective in controlling this disease.

In recent years, virology studies of influenza virus illustrated the replication process of the virus and some molecular targets have been identified for drug intervention. Neuraminidase (NA), a glycoprotein embedded in the viral envelope, plays a role at the final stage of infection when NA cleaves sialic acid from cell surface and progeny virions facilitating virus release from an infected cell. Studies with neuraminidase-deficient influenza virus have shown that the mutant virus is still infective but the budding virus particles form aggregates or remain bound to the infected cell surface. Compounds that inhibit neuraminidase may be able to protect the host from viral infection and retard its propagation by preventing spread of the virus among cells and from the site of infection.

Over the last two decades, a number of classes of NA inhibitors have been developed and have shown to be somewhat effective in controlling influenza infection. Zanamivir (Relenza) and Oseltamivir (Tamiflu) have been approved by FDA for treatment and prevention of influenza. Zanamivir is an inhibitor of both A and B forms of neuraminidase, but this highly polar compound requires administration by oral inhalation to achieve efficacy due to its low lipophilicity and low oral bioavailability. The ester oseltamivir is a prodrug converted after oral intake to its active form, the carboxylic acid. Oseltamivir is also an inhibitor of both A and B forms of neuraminidase, but it has been reported to cause nausea and vomiting. In addition, psychiatric effects in children have been reported after treatment with Tamiflu, prompting FDA to recommend adding warnings on the label about such possible side effects. Also, drug resistance is expected with widespread use of both of the agents and the emergence of mutant viral strains mandates new chemical entities.

### SUMMARY OF THE INVENTION

The present invention provides compounds of formula I

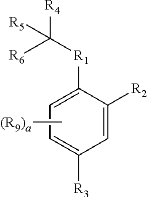

wherein: R₁ is O, S, NH, or NR₇; R₂ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NH)NH₂, C(═NR₇)N(R₇)₂, or NR₈; R₃ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; R₄ is an amino acid R group; R₅ is H or C₁₋₆alkyl; or R₄ and R₅, taken together, are (CH₂)ₙ, where n is 2 to 6; R₆ is COOH, sulfonate, phosphonate, or phosphate; each R₇ is, independently, C₁₋₆alkyl; each R₈ is, independently, C₁₋₆alkyl or H; each R₉ is, independently, halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; and a is 0 to 3; or a pharmaceutically acceptable salt thereof; provided that the compound is not dinitrophenyl isoleucine, nitrocyanophenyl isoleucine, dinitrophenyl valine, nitrocyanophenyl valine, dinitrophenyl alanine, nitrocyanophenyl alanine, dinitrophenyl glutamine, dinitrophenyl methionine, N-2,4-dinitrophenyl-DL-methionine sulfone, dinitrophenyl serine, dinitrophenyl arginine, dinitrophenyl glycine, and dinitrophenyl tryptophan.

The present invention also provides compounds of formula II

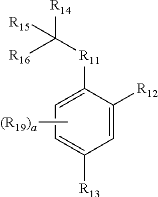

wherein: R₁₁ is O-phenyl-CH₂—; R₁₂ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NH)NH₂, C(═NR₁₇)N(R₁₇)₂, or NR₁₈; R₁₃ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; R₁₄ is N(R₂₂)₂; each R₂₂ is, independently, hydrogen, halogen, C₁₋₆alkyl, or hydroxyl; R₁₅ is H or C₁₋₆alkyl; R₁₆ is COOH, sulfonate, phosphonate, or phosphate; each R₁₇ is, independently, C₁₋₆alkyl; each R₁₈ is, independently, C₁₋₆alkyl or H; each R₁₉ is, independently, halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; and a is 0 to 3; or a pharmaceutically acceptable salt thereof; provided that the compound is not dinitrophenyl tyrosine.

The present invention also provides pharmaceutical compositions comprising a compound, or a pharmaceutically acceptable salt thereof, of formula I or formula II and a pharmaceutically acceptable carrier.

The present invention also provides methods of inhibiting neuraminidase in a cell comprising contacting the cell with an inhibitory effective amount of a compound of formula I, or a pharmaceutically acceptable salt thereof,

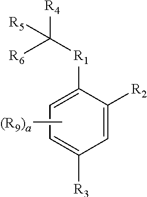

wherein: R₁ is O, S, NH, or NR₇; R₂ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NH)NH₂, C(═NR₇)N(R₇)₂, or NR₈; R₃ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; R₄ is an amino acid R group; R₅ is H or C₁₋₆alkyl; or R₄ and R₅, taken together, are (CH₂)ₙ, where n is 2 to 6; R₆ is COOH, sulfonate, phosphonate, or phosphate; each R₇ is, independently, C₁₋₆alkyl; each R₈ is, independently, C₁₋₆alkyl or H; each R₉ is, independently, halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; and a is 0 to 3.

The present invention also provides methods of treating influenza, Pseudomonas aeruginosa, or Bacteroides fragilis infection in a mammal comprising contacting the mammal with a therapeutically effective amount of a compound of formula I, or a pharmaceutically acceptable salt thereof,

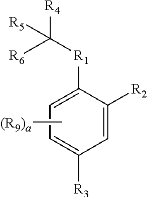

wherein: R₁ is O, S, NH, or NR₇; R₂ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NH)NH₂, C(═NR₇)N(R₇)₂, or NR₈; R₃ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; R₄ is an amino acid R group; R₅ is H or C₁₋₆alkyl; or R₄ and R₅, taken together, are (CH₂)ₙ, where n is 2 to 6; R₆ is COOH, sulfonate, phosphonate, or phosphate; each R₇ is, independently, C₁₋₆alkyl; each R₈ is, independently, C₁₋₆alkyl or H; each R₉ is, independently, halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; and a is 0 to 3.

The present invention also provides methods of inhibiting neuraminidase in a cell comprising contacting the cell with an inhibitory effective amount of a compound of formula II, or dinitrophenyl proline, or dinitrophenyl histidine, or a pharmaceutically acceptable salt thereof,

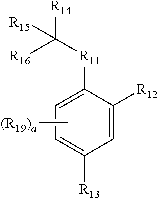

wherein: R₁₁ is O-phenyl-CH₂—; R₁₂ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NH)NH₂, C(═NR₁₇)N(R₁₇)₂, or NR₁₈; R₁₃ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; R₁₄ is N(R₂₂)₂; each R₂₂ is, independently, hydrogen, halogen, C₁₋₆alkyl, or hydroxyl; R₁₅ is H or C₁₋₆alkyl; R₁₆ is COOH, sulfonate, phosphonate, or phosphate; each R₁₇ is, independently, C₁₋₆alkyl; each R₁₈ is, independently, C₁₋₆alkyl or H; each R₁₉ is, independently, halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; and a is 0 to 3.

The present invention also provides methods of treating influenza, Pseudomonas aeruginosa, or Bacteroides fragilis infection in a mammal comprising contacting the mammal with a therapeutically effective amount of a compound of formula II, or dinitrophenyl proline, or dinitrophenyl histidine, or a pharmaceutically acceptable salt thereof,

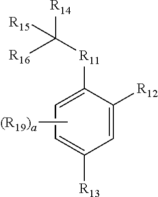

wherein: R₁₁ is O-phenyl-CH₂—; R₁₂ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NH)NH₂, C(═NR₁₇)N(R₁₇)₂, or NR₁₈; R₁₃ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; R₁₄ is N(R₂₂)₂; each R₂₂ is, independently, hydrogen, halogen, C₁₋₆alkyl, or hydroxyl; R₁₅ is H or C₁₋₆alkyl; R₁₆ is COOH, sulfonate, phosphonate, or phosphate; each R₁₇ is, independently, C₁₋₆alkyl; each R₁₈ is, independently, C₁₋₆alkyl or H; each R₁₉ is, independently, halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; and a is 0 to 3.

The present invention also provides compounds of formula I, formula II, or dinitrophenyl proline, or dinitrophenyl histidine, or a pharmaceutically acceptable salt thereof, for inhibiting neuraminidase in a cell.

The present invention also provides compounds of formula I, formula II, or dinitrophenyl proline, or dinitrophenyl histidine, or a pharmaceutically acceptable salt thereof, for treating influenza, Pseudomonas aeruginosa, or Bacteroides fragilis infection.

The present invention also provides compounds of formula I, formula II, or dinitrophenyl proline, or dinitrophenyl histidine, or a pharmaceutically acceptable salt thereof, for use in the manufacture of a medicament for the treatment of influenza, Pseudomonas aeruginosa, or Bacteroides fragilis infection.

### DESCRIPTION OF EMBODIMENTS

As used herein, the term “alkyl” refers to a group containing one to six carbon atoms (i.e, C₁₋₆) or containing one to three carbon atoms (i.e, C₁₋₃). It may be straight-chained or branched. Examples include, but are not limited to, methyl, ethyl, propyl, isopropyl, butyl, sec-butyl, isobutyl, tert-butyl, pentyl, neopentyl, hexyl and the like.

As used herein, the term “alkenyl” refers to an alkyl group having one or more double carbon-carbon bonds. Examples of alkenyl groups include, but are not limited to, ethenyl, propenyl, cyclohexenyl, and the like.

As used herein, the term “alkynyl” refers to an alkyl group having one or more triple carbon-carbon bonds. Examples of alkynyl groups include, but are not limited to, ethynyl, propynyl, and the like.

As used herein, the term “alkoxy” refers to an alkyl group as defined above with the indicated number of carbon atoms attached through an oxygen bridge. Examples of alkoxy groups include, but are not limited to, methoxy, ethoxy, n-propoxy, isopropoxy, n-butoxy, isobutoxy, t-butoxy, n-pentoxy, isopentoxy, cyclopropylmethoxy, allyloxy, and propargyloxy, or any subset thereof.

As used herein, the term “halogen” refers to fluoro, chloro, bromo, and iodo.

As used herein, the phrase “pharmaceutically acceptable” refers to those compounds and/or compositions that are suitable for use in contact with the tissues of human beings and other animals or mammals.

As used herein, the phrase “pharmaceutically acceptable salt” refers to derivatives of the disclosed compounds wherein the compound is modified by making an acid or base salt thereof. Examples of acid addition salts include, but are not limited to, salts formed with hydrochloric, hydrobromic, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, tartaric, acetic, propanoic, butanoic, malonic, glucuronic, and lactobionic acids. The pharmaceutically acceptable salts of the present invention can be synthesized from the compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. In addition, nonaqueous media like ether, ethyl acetate, ethanol, isopropanol, or acetonitrile can be used.

As used herein, the phrase “amino acid R group” refers to any R group that is comprised within an amino acid residue. Amino acid residues are well known to the skilled artisan and numerous such amino acid residues, or variants thereof, are commercially available. For example, a typical amino acid comprises the following backbone core:

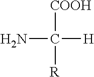

It is the amino acid R group depicted in the backbone that, thus, distinguishes one amino acid from another. In some embodiments, the amino acid R group is: a nonpolar amino acid R group, a polar uncharged amino acid R group, a positively charged amino acid R group, or a negatively charged amino acid R group, or any combination thereof (see, for example, Organic Chemistry, 2^{nd} Edition, John Wiley & Sons, New York, p. 101, which is incorporated herein by reference). Suitable amino acid R groups include, but are not limited to, an isoleucine R group, a valine R group, an alanine R group, a glutamine R group, a methionine R group, a serine R group, an arginine R group, a glycine R group, and a tryptophan R group. The attachment of the amino acid R group (R₄) to the remainder of the compound depicted in formula I is generally via the nitrogen in the amino acid backbone (i.e., one hydrogen within the NH₂ moiety in the amino acid backbone is replaced with the R₂/R₃ substituted phenyl depicted in formula I; the NH moiety of the amino acid backbone, which has had one hydrogen removed, serves as the R₁ group depicted in formula I).

In some embodiments, the amino acid R group can be further substituted. For example, for the methionine R group, the sulfur atom can be further substituted with, for example, ═O, to create S(═O)₂ (i.e., N-2,4-dinitrophenyl-DL-methionine sulfone).

In some embodiments, a suitable amino acid R group is a tyrosine R group. In this embodiment, the oxygen within the CH₂-phenyl-OH group of the tyrosine residue is attached to the R₁₂/R₁₃ substituted phenyl depicted in formula II, as shown below. Thus, the hydroxyl group (upon removal of the hydrogen) of the amino acid R group of tyrosine serves as the R₁₁ group depicted in formula II.

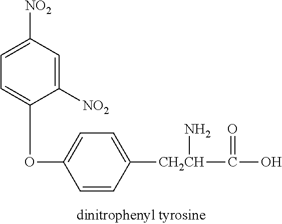

In some embodiments, a suitable amino acid R group is a histidine R group. In this embodiment, two R₂/R₃ substituted phenyls are attached to the histidine R group. The first R₂/R₃ substituted phenyl is attached to the nitrogen in the amino acid backbone of histidine (i.e., one hydrogen within the NH₂ moiety in the amino acid backbone is replaced with the R₂/R₃ substituted phenyl; the NH moiety of the amino acid backbone, which has had one hydrogen removed, serves as the R₁ group depicted in Formula I). The second R₂/R₃ substituted phenyl is attached to the nitrogen that is double bonded to the carbon within the ring of the histidine residue.

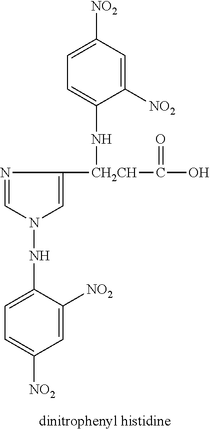

In some embodiments, a suitable amino acid R group is a proline R group. In this embodiment, the R₂/R₃ substituted phenyl is attached directly to the nitrogen within the proline as shown below.

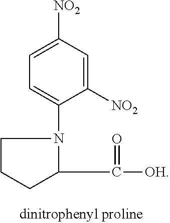

The present invention provides compounds of formula I

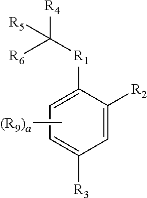

wherein:

R₁ is O, S, NH, or NR₇;

R₂ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NH)NH₂, C(═NR₇)N(R₇)₂, or NR₈;

R₃ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈;

R₄ is an amino acid R group;

R₅ is H or C₁₋₆alkyl;
or R₄ and R₅, taken together, are (CH₂)ₙ, where n is 2 to 6;

R₆ is COOH, sulfonate, phosphonate, or phosphate;

each R₇ is, independently, C₁₋₆alkyl;

each R₈ is, independently, C₁₋₆alkyl or H;

each R₉ is, independently, halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; and

a is 0 to 3;

or a pharmaceutically acceptable salt thereof;

provided that the compound is not dinitrophenyl isoleucine, nitrocyanophenyl isoleucine, dinitrophenyl valine, nitrocyanophenyl valine, dinitrophenyl alanine, nitrocyanophenyl alanine, dinitrophenyl glutamine, dinitrophenyl methionine, N-2,4-dinitrophenyl-DL-methionine sulfone, dinitrophenyl serine, dinitrophenyl arginine, dinitrophenyl glycine, and dinitrophenyl tryptophan.

In some embodiments, R₁ is O, NH, or NR₇. In some embodiments, R₁ is NH or NR₇. In some embodiments, R₁ is NH. In some embodiments, R₁ is NR₇.

In some embodiments, R₂ is halogen, CN, NO₂, or C₁₋₆alkyl. In some embodiments, R₂ is CN, NO₂, or C₁₋₆alkyl. In some embodiments, R₂ is halogen, NO₂, or C₁₋₆alkyl. In some embodiments, R₂ is halogen, CN, or C₁₋₆alkyl. In some embodiments, R₂ is halogen, CN, or NO₂. In some embodiments, R₂ is NO₂ or C₁₋₆alkyl. In some embodiments, R₂ is CN or C₁₋₆alkyl. In some embodiments, R₂ is CN or NO₂. In some embodiments, R₂ is halogen or C₁₋₆alkyl. In some embodiments, R₂ is halogen or NO₂. In some embodiments, R₂ is halogen or CN. In some embodiments, R₂ is halogen. In some embodiments, R₂ is CN. In some embodiments, R₂ is NO₂. In some embodiments, R₂ is C₁₋₆alkyl. In some embodiments, R₂ is halogen, CN, NO₂, or C₁₋₃alkyl. In some embodiments, R₂ is CN, NO₂, or C₁₋₃alkyl. In some embodiments, R₂ is halogen, NO₂, or C₁₋₃alkyl. In some embodiments, R₂ is halogen, CN, or C₁₋₃alkyl. In some embodiments, R₂ is NO₂ or C₁₋₃alkyl. In some embodiments, R₂ is CN or C₁₋₃alkyl. In some embodiments, R₂ is halogen or C₁₋₃alkyl. In some embodiments, R₂ is C₁₋₃alkyl.

In some embodiments, R₃ is halogen, CN, NO₂, or C₁₋₆alkyl. In some embodiments, R₃ is CN, NO₂, or C₁₋₆alkyl. In some embodiments, R₃ is halogen, NO₂, or C₁₋₆alkyl. In some embodiments, R₃ is halogen, CN, or C₁₋₆alkyl. In some embodiments, R₃ is halogen, CN, or NO₂. In some embodiments, R₃ is NO₂ or C₁₋₆alkyl. In some embodiments, R₃ is CN or C₁₋₆alkyl. In some embodiments, R₃ is CN or NO₂. In some embodiments, R₃ is halogen or C₁₋₆alkyl. In some embodiments, R₃ is halogen or NO₂. In some embodiments, R₃ is halogen or CN. In some embodiments, R₃ is halogen. In some embodiments, R₃ is CN. In some embodiments, R₃ is NO₂. In some embodiments, R₃ is C₁₋₆alkyl. In some embodiments, R₃ is halogen, CN, NO₂, or C₁₋₃alkyl. In some embodiments, R₃ is CN, NO₂, or C₁₋₃alkyl. In some embodiments, R₃ is halogen, NO₂, or C₁₋₃alkyl. In some embodiments, R₃ is halogen, CN, or C₁₋₃alkyl. In some embodiments, R₃ is NO₂ or C₁₋₃alkyl. In some embodiments, R₃ is CN or C₁₋₃alkyl. In some embodiments, R₃ is halogen or C₁₋₃alkyl. In some embodiments, R₃ is C₁₋₃alkyl.

In some embodiments, R₄ is a nonpolar amino acid R group, a polar uncharged amino acid R group, or a positively charged amino acid R group. In some embodiments, R₄ is a polar uncharged amino acid R group, or a positively charged amino acid R group. In some embodiments, R₄ is a nonpolar amino acid R group, or a positively charged amino acid R group. In some embodiments, R₄ is a nonpolar amino acid R group, or a polar uncharged amino acid R group. In some embodiments, R₄ is a nonpolar amino acid R group. In some embodiments, R₄ is a polar uncharged amino acid R group. In some embodiments, R₄ is a positively charged amino acid R group.

In some embodiments, R₅ is H or C₁₋₃alkyl. In some embodiments, R₅ is H. In some embodiments, R₅ is C₁₋₃alkyl.

In some embodiments, R₆ is COOH, phosphonate, or phosphate. In some embodiments, R₆ is COOH, sulfonate, or phosphate. In some embodiments, R₆ is COOH, sulfonate, or phosphonate. In some embodiments, R₆ is COOH or sulfonate. In some embodiments, R₆ is COOH or phosphonate. In some embodiments, R₆ is COOH or phosphate. In some embodiments, R₆ is COOH.

In some embodiments, each R₇ is, independently, C₁₋₃alkyl.

In some embodiments, each R₈ is, independently, C₁₋₃alkyl or H. In some embodiments, each R₈ is, independently, C₁₋₃alkyl. In some embodiments, each R₈ is, independently, H.

In some embodiments, R₉ is halogen, CN, NO₂, or C₁₋₆alkyl. In some embodiments, R₉ is CN, NO₂, or C₁₋₆alkyl. In some embodiments, R₉ is halogen, NO₂, or C₁₋₆alkyl. In some embodiments, R₉ is halogen, CN, or C₁₋₆alkyl. In some embodiments, R₉ is halogen, CN, or NO₂. In some embodiments, R₉ is NO₂ or C₁₋₆alkyl. In some embodiments, R₉ is CN or C₁₋₆alkyl. In some embodiments, R₉ is CN or NO₂. In some embodiments, R₉ is halogen or C₁₋₆alkyl. In some embodiments, R₉ is halogen or NO₂. In some embodiments, R₉ is halogen or CN. In some embodiments, R₉ is halogen. In some embodiments, R₉ is CN. In some embodiments, R₉ is NO₂. In some embodiments, R₉ is C₁₋₆alkyl. In some embodiments, R₉ is halogen, CN, NO₂, or C₁₋₃alkyl. In some embodiments, R₉ is CN, NO₂, or C₁₋₃alkyl. In some embodiments, R₉ is halogen, NO₂, or C₁₋₃alkyl. In some embodiments, R₉ is halogen, CN, or C₁₋₃alkyl. In some embodiments, R₉ is NO₂ or C₁₋₃alkyl. In some embodiments, R₉ is CN or C₁₋₃alkyl. In some embodiments, R₉ is halogen or C₁₋₃alkyl. In some embodiments, R₉ is C₁₋₃alkyl.

In some embodiments, a is 0 to 3 or 1 to 3. In some embodiments, a is 0. In some embodiments, a is 1.

In some embodiments, R₁ is O, NH, or NR₇; R₂ is halogen, CN, NO₂, C₁₋₆alkyl, CONHR₇, NHCOR₇, COOR₇, C═NOH, C(═NH)NH₂, C(═NR₇)N(R₇)₂, or NR₈; R₃ is halogen, CN, NO₂, C₁₋₆alkyl, CONHR₇, NHCOR₇, COOR₇, C═NOH, C(═NH)NH₂, C(═NR₇)N(R₇)₂, or NR₈; R₄ is an amino acid R group; R₅ is H or C₁₋₆alkyl; R₆ is COOH; each R₇ is, independently, C₁₋₆alkyl; each R₈ is, independently, C₁₋₆alkyl or H; a is 0 to 3; and each R₉ is, independently, halogen, CN, NO₂, or C₁₋₆alkyl.

In some embodiments, R₁ is NH or NR₇; R₂ is halogen, CN, NO₂, or C₁₋₆alkyl; R₃ is halogen, CN, NO₂, or C₁₋₆alkyl; R₄ is an amino acid R group; R₅ is H or C₁₋₆alkyl; R₆ is COOH; R₇ is C₁₋₆alkyl; and a is 0.

In some embodiments, R₁ is NH; R₂ is CN or NO₂; R₃ is NO₂; R₄ is an isoleucine R group, a valine R group, an alanine R group, a glutamine R group, a methionine R group, a serine R group, an arginine R group, a glycine R group, or a tryptophan R group; R₅ is H; R₆ is COOH; a is 1 to 3; and each R₉ is, independently, halogen, CN, NO₂, or C₁₋₆alkyl.

The present invention also provides compounds of formula II

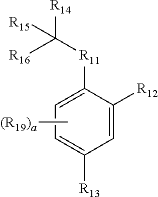

wherein:

R₁₁ is O-phenyl-CH₂—;

R₁₂ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NH)NH₂, C(═NR₁₇)N(R₁₇)₂, or NR₁₈;

R₁₃ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈;

R₁₄ is N(R₂₂)₂;

each R₂₂ is, independently, hydrogen, halogen, C₁₋₆alkyl, or hydroxyl;

R₁₅ is H or C₁₋₆alkyl;

R₁₆ is COOH, sulfonate, phosphonate, or phosphate;

each R₁₇ is, independently, C₁₋₆alkyl;

each R₁₈ is, independently, C₁₋₆alkyl or H;

each R₁₉ is, independently, halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; and

a is 0 to 3;

or a pharmaceutically acceptable salt thereof;

provided that the compound is not dinitrophenyl tyrosine.

In some embodiments, R₁₁ is O-phenyl-CH₂—; R₁₂ is halogen, CN, NO₂, or C₁₋₆alkyl; R₁₃ is halogen, CN, NO₂, or C₁₋₆alkyl; R₁₄ is N(R₂₂)₂; each R₂₂ is, independently, hydrogen or C₁₋₃alkyl; R₁₅ is H or C₁₋₆alkyl; R₁₆ is COOH; each R₁₉ is, independently, halogen, CN, NO₂, or C₁₋₆alkyl; and a is 0 to 3.

In some embodiments, R₁₁ is O-phenyl-CH₂—; R₁₂ is CN or NO₂; R₁₃ is CN or NO₂; R₁₄ is NH₂; R₁₅ is H or C₁₋₆alkyl; R₁₆ is COOH; each R₁₉ is, independently, halogen, CN, NO₂, or C₁₋₆alkyl; and a is 1 to 3.

In some embodiments, R₁₁ is O-phenyl-CH₂—; R₁₂ is CN or NO₂; R₁₃ is CN or NO₂; R₁₄ is NH₂; R₁₅ is H; R₁₆ is COOH; and a is 0.

In some embodiments, R₁₁ is O-phenyl-CH₂—; R₁₂ is halogen, CN, NO₂, C₁₋₆alkyl, CONHR₁₇, NHCOR₁₇, COOR₁₇, C═NOH, C(═NH)NH₂, C(═NR₁₇)N(R₁₇)₂, or NR₁₈; R₁₃ is halogen, CN, NO₂, C₁₋₆alkyl, CONHR₁₇, NHCOR₁₇, COOR₁₇, C═NOH, C(═NR₁₇)N(R₁₇)₂, or NR₁₈; R₁₄ is N(R₂₂)₂; each R₂₂ is, independently, hydrogen, halogen, C₁₋₆alkyl, or hydroxyl; R₁₅ is H or C₁₋₆alkyl; R₁₆ is COOH, sulfonate, phosphonate, or phosphate; each R₁₇ is, independently, C₁₋₆alkyl; each R₁₈ is, independently, C₁₋₆alkyl or H; each R₁₉ is, independently, halogen, CN, NO₂, C₁₋₆alkyl, CONHR₁₇, NHCOR₁₇, COOR₁₇, C═NOH, C(═NR₁₇)N(R₁₇)₂, or NR₁₈; and a is 0 to 3.

The compounds of formula I or formula II, or dinitrophenyl proline, or dinitrophenyl histidine, can be obtained commercially or synthesized according to routine methods using commercially available starting compounds. Representative methods are described in the Examples.

The present invention also provides pharmaceutical compositions comprising a compound, or a pharmaceutically acceptable salt thereof, of any one of the compounds of formula I or formula II described herein and a pharmaceutically acceptable carrier.

The present invention also provides methods of inhibiting neuraminidase in a cell comprising contacting the cell with an inhibitory effective amount of a compound of formula I, or a pharmaceutically acceptable salt thereof, described herein. In these methods, the compound can be dinitrophenyl isoleucine, nitrocyanophenyl isoleucine, dinitrophenyl valine, nitrocyanophenyl valine, dinitrophenyl alanine, nitrocyanophenyl alanine, dinitrophenyl glutamine, dinitrophenyl methionine, N-2,4-dinitrophenyl-DL-methionine sulfone, dinitrophenyl serine, dinitrophenyl arginine, dinitrophenyl glycine, and/or dinitrophenyl tryptophan.

The present invention also provides methods of inhibiting neuraminidase in a cell comprising contacting the cell with an inhibitory effective amount of a compound of formula II, or dinitrophenyl proline, or dinitrophenyl histidine, or a pharmaceutically acceptable salt thereof, described herein. In these methods, the compound can be dinitrophenyl tyrosine.

In some embodiments, the cell is an isolated cell. In some embodiments, the cell forms a part of a tissue. In other embodiments, the cell is within a mammal.

The present invention also provides methods of treating influenza, Pseudomonas aeruginosa, or Bacteroides fragilis infection in a mammal comprising contacting the mammal with a therapeutically effective amount of a compound of formula I, or a pharmaceutically acceptable salt thereof, described herein. In these methods, the compound can be dinitrophenyl isoleucine, nitrocyanophenyl isoleucine, dinitrophenyl valine, nitrocyanophenyl valine, dinitrophenyl alanine, nitrocyanophenyl alanine, dinitrophenyl glutamine, dinitrophenyl methionine, N-2,4-dinitrophenyl-DL-methionine sulfone, dinitrophenyl serine, dinitrophenyl arginine, dinitrophenyl glycine, and/or dinitrophenyl tryptophan.

The present invention also provides methods of treating influenza, Pseudomonas aeruginosa, or Bacteroides fragilis infection in a mammal comprising contacting the mammal with a therapeutically effective amount of a compound of formula II, or dinitrophenyl proline, or dinitrophenyl histidine, or a pharmaceutically acceptable salt thereof, described herein. In these methods, the compound can be dinitrophenyl tyrosine.

In some embodiments, the influenza is influenza A virus. In some embodiments, the influenza is influenza B virus. In some embodiments, the influenza is avian influenza virus.

In some embodiments, the mammal being treated is in need of such treatment. In such mammals, the mammal may have been diagnosed as having influenza, Pseudomonas aeruginosa, or Bacteroides fragilis infection, or suspected of having influenza, Pseudomonas aeruginosa, or Bacteroides fragilis infection.

The present invention also provides compounds of formula I, formula II, or dinitrophenyl proline, or dinitrophenyl histidine, or a pharmaceutically acceptable salt thereof, for inhibiting neuraminidase in a cell.

The present invention also provides compounds of formula I, formula II, or dinitrophenyl proline, or dinitrophenyl histidine, or a pharmaceutically acceptable salt thereof, for treating influenza, Pseudomonas aeruginosa, or Bacteroides fragilis infection.

The present invention also provides compounds of formula I, formula II, or dinitrophenyl proline, or dinitrophenyl histidine, or a pharmaceutically acceptable salt thereof, for use in the manufacture of a medicament for the treatment of influenza, Pseudomonas aeruginosa, or Bacteroides fragilis infection.

The pharmaceutical compositions can be in unit dosage form such that the composition is divided into unit doses containing appropriate quantities of the active ingredient. The unit dosage form can be in the form of a tablet, capsule, or powder. Alternately, the pharmaceutical compositions can be oral pharmaceutical compositions comprising a liquid.

Methods of preparing dosage forms are known to those skilled in this art and are disclosed in, for example, Remington's Pharmaceutical Sciences, Mack Publishing Company, Easton, Pa., 15th Edition, 1975.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration.

The term composition is intended to include the formulation of the active ingredient or a pharmaceutically acceptable salt with a pharmaceutically acceptable carrier, and optionally other ingredients. For preparing pharmaceutical compositions from the compounds of this invention, inert, pharmaceutically acceptable carriers can be either solid or liquid. For example, this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Solid form compositions include powders, tablets, dispersible granules, capsules, cachets, and suppositories. A solid carrier can be one or more substances, which may also act as diluents, flavoring agents, solubilizers, lubricants, suspending agents, binders, or tablet disintegrating agents.

A suitable composition can be administered to mammals by aerosol delivery to the lungs to prevent or decrease the intrapleural spread of influenza infection. The compound or composition can be administered in an amount sufficient to produce this anti-viral effect without causing pulmonary side effects. The useful dose range of a suitable compound or composition can be readily determined by one of skill in the art through routine testing. One skilled in the art recognizes that the starting dose range for aerosol delivery depends upon a number of factors, including the mammals's breathing characteristics, the type of device used (metered dose inhaler or nebulizer for aerosols, or dry powder inhaler as an alternative to aerosol delivery), the device's efficiency in depositing aerosolized drug in the lung relative to the fraction that is swallowed, the rate of clearance of the compound from lung epithelium by that route of administration, as well as the necessary extracellular concentration of the compound required to produce a therapeutically effective intracellular concentration in lung epithelial and related cells. Because of these numerous variables, a suitable method of dose ranging is to titrate the dose to effect, e.g., to employ a dose that is found to effectively reduce (or preclude the detection or elevation of) influenza viral burden, as measured in combined nose and throat swab specimens or nasal washes by a rapid and sensitive quantitative PCR assay (van Elden et al. Simultaneous Detection of Influenza Viruses A and B Using Real-Time Quantitative PCR. J. Clin. Microbiol., 2001, 39, 196-200). In this case, although only two samples, before and after drug administration, are necessary to compare the pretreatment viral burden with the post-treatment level, it is suitable to measure the virus levels at timed intervals so that the dosing interval can be adjusted to maintain a persistently suppressed viral level without producing clinically important side effects.

Solid or liquid particulate forms of the active compound prepared for practicing the present invention include particles of respirable size: that is, particles of a size sufficiently small to pass through the mouth and larynx upon inhalation and into the bronchi and alveoli of the lungs. In general, particles ranging from about 1 to 10 microns in size are within the respirable range, with an optimal range of 1-5 microns. The therapeutic compositions can be administered by direct inhalation into the respiratory system for delivery as a mist or other aerosol or dry powder. Particles of non-respirable size which are included in the aerosol tend to be deposited in the throat and swallowed; thus the quantity of non-respirable particles in the aerosol is preferably minimized.

Aerosols of liquid particles can be produced by any suitable means, such as inhalatory delivery systems. One is a traditional nebulizer which works in a mechanism similar to the familiar perfume atomizer. The airborne particles are generated by a jet of air from either a compressor or compressed gas cylinder-passing through the device (pressure-driven aerosol nebulizer). In addition, newer forms utilize an ultrasonic nebulizer by vibrating the liquid at speed of up to about 1 MHz. See, e.g., U.S. Pat. No. 4,501,729, the contents of which are incorporated by reference. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier. The carrier is typically water (and most preferably sterile, pyrogen-free water) or a dilute aqueous alcoholic solution, preferably made isotonic but may be hypertonic with body fluids by the addition of, for example, sodium chloride. Optional additives include preservatives if the formulation is not made sterile, for example, methyl hydroxybenzoate, as well as antioxidants, flavoring agents, volatile oils, buffering agents and surfactants, which are normally used in the preparation of pharmaceutical compositions.

Aerosols of solid particles can be likewise produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for mammal administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the compound, a suitable powder diluent, such as lactose, and an optional surfactant. A second type of illustrative aerosol generator comprises a metered dose inhaler. Metered dose inhalers are pressurized aerosol dispensers, typically containing a suspension or solution formulation of the compound in a liquified propellant. During use these devices discharge the formulation through a valve, adapted to deliver a metered volume, from 10 to 22 microliters to produce a fine particle spray containing the compound. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

Any propellant may be used in carrying out the present invention, including both chlorofluorocarbon-containing propellants and non-chlorofluorocarbon-containing propellants. Fluorocarbon aerosol propellants that may be employed in carrying out the present invention including fluorocarbon propellants in which all hydrogen are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Examples of such propellants include, but are not limited to, CF₃CHFCF₂ and the like. A stabilizer such as a fluoropolymer may optionally be included in formulations of fluorocarbon propellants, such as described in U.S. Pat. No. 5,376,359 to Johnson.

Compositions containing respirable dry particles of micronized compounds may be prepared by grinding the dry active compound, with e.g., a mortar and pestle or other appropriate grinding device, and then passing the micronized composition through a 400 mesh screen to break up or separate out large agglomerates.

The aerosol, whether formed from solid or liquid particles, may be produced by the aerosol generator at a rate of from about 10 to 150 liters per minute. Aerosols containing greater amounts of medicament may be administered more rapidly. Typically, each aerosol may be delivered to the mammal for a period from about 30 seconds to about 20 minutes, or of about 1 to 5 minutes.

The particulate composition comprising the compound may optionally contain a carrier which serves to facilitate the formation of an aerosol. A suitable carrier is lactose, which may be blended with the active compound in any suitable ratio. For example, hydroxychloroquine sulfate is a colorless crystalline solid which is readily soluble in water. Inhaled liquid forms may be formulated to contain such additives as are typically used in such pharmaceutical preparations, including, but not limited to an acceptable excipient and/or surfactant. A therapeutic composition of hydroxychloroquine may be pre-formulated in liquid form, or prepared for the addition of a suitable carrier, like sterile water or physiological saline, immediately prior to use. The aerosol containing hydroxychloroquine typically contain a propellant, especially a fluorocarbon propellant. See Remington's, chapter 92. A particularly useful composition of hydroxychloroquine is formulated in a nebulizer, e.g., as disclosed by Charous in U.S. Pat. No. 6,572,858. For the preparation of hydroxychloroquine in inhaled powder form, the compound is finely divided, or micronized to enhance effectiveness, and admixed with a suitable filler. Inhaled powders may contain a bulking agent and/or stabilizer, as described hereinabove. Id., chapter 88. An insufflator (powder blower) may be employed to administer the fine powder.

The compounds also may be formulated advantageously as nasal drops or sprays. These preparations contain the active ingredient in a effective amount and concentration, i.e. one ensuring a protective action against virus infections of the above mentioned kind. Accordingly, the nasal preparations of the invention contain the active ingredient in a concentration suitable for administration of at least 2 mg of active ingredient each time. The upper limit is 4% (w/v) and the lower limit, because single administration can be made not only by once only nasal application, but also by repeated application once or more than once after the preparation has dried, is about 0.2% (w/v), corresponding to 1 ml of a ready-for-use nasal preparation for the above minimum dose. Nasal drops have in particular a concentration between 1% and 2% and sprays a concentration between 0.2 and 1% of active ingredient. In both formulations the solvent can be water. The aqueous solutions optionally contain conventional pharmaceutically acceptable excipients for stabilizing the active ingredient, as well as for buffering, preserving and/or lowering the surface tension, and they can be made isotonic in conventional manner, e.g. with sodium chloride or buffer solutions.

The invention relates in particular to spray bottles filled with the above solutions and to similar containers suitable for the intranasal application of such solutions.

The quantity of the compound to be administered will vary depending upon the mammal or human being treated and will vary from about 100 ng/kg of body weight to about 100 mg/kg of body weight per day, from about 1 μg/kg of body weight to about 10 mg/kg of body weight per day, from about 10 μg/kg of body weight to about 1 mg/kg of body weight per day, or from about 100 μg/kg of body weight to about 500 μg/kg of body weight per day. Dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention, and can adjust the starting dosage as needed depending upon the status of the infection.

In order that the invention disclosed herein may be more efficiently understood, examples are provided below. It should be understood that these examples are for illustrative purposes only and are not to be construed as limiting the invention in any manner.

### EXAMPLES

The compounds described herein can be either synthesized by the following Scheme 1 or obtained through commercial sources.

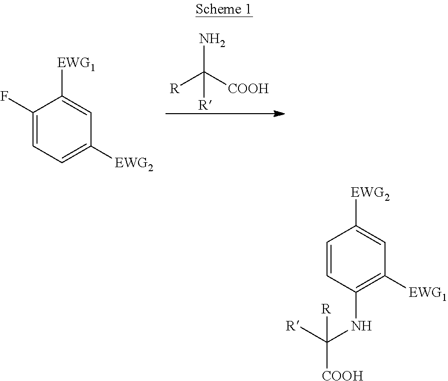

EWG represent electron withdrawing groups (see R₂, R₃, R₁₂, and R₁₃ described herein). Numerous such starting compounds are available from several venders. For example, DNP-L-Alanine (Catalog #D7254), DNP-L-Valine (Catalog #D2380), DNP-L-Arginine (Catalog #D8129), N-2,4-DNP-DL-Methionine sulfone (Catalog #D0630), N-2,4-DNP-L-Tryptophan (Catalog #D2005), DNP-L-Glutamine (Catalog #D9379), DNP-L-Proline (Catalog #D1505), DNP-Glycine (Catalog #D9504), DNP-L-Serine (Catalog #D1755), N,N′-di(DNP)-L-Tyrosine (Catalog #D2255) can be obtained from Sigma.

### Example 1

### (2S,3S)-2-(5-fluoro-2,4-dinitrophenylamino)-3-methylpentanoic acid (1)

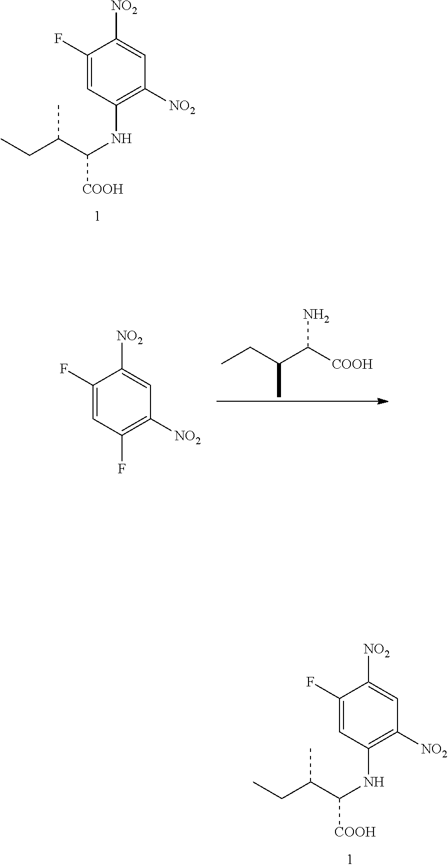

To a 25 ml of round bottom flask was added a solution of L-Isoleucine (135.0 mg, 1.03 mmol, 98%) and sodium bicarbonate (210.1 mg, 2.5 mmol) in water (6 ml) followed by 1,5-difluoro-2,4-dinitrobenzene (210.4 mg, 1.0 mmol, 97%) at room temperature. The reaction mixture was stirred for 90 minutes at room temperature. TLC analysis suggested that the starting material was consumed and, at which time, the reaction mixture was acidified with HCl (1 N) solution to pH ˜3. The yellow N-arylated amino acid, collected via filtration, was washed with water and ether sequentially and air-dried to yield 215.3 mg (68%) of the desired product. MS (m/z) 314.1 (M⁺−1).

### Example 2

### (2S,3S)-2-(2-cyano-4-nitrophenylamino)-3-methylpentanoic acid (2)

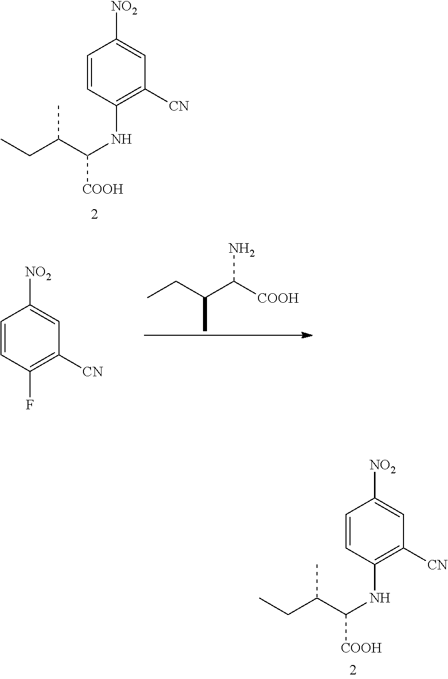

L-isoleucine (10.0 mg, 0.076 mmol) in 4 ml of borate buffer solution (pH 8.2) was added 2-fluoro-5-nitro-benzonitrile (11.3 mg, 0.068 mmol) in 60 μL of acetonitrile. The reaction mixture was heated at 50° C. for 2-3 hours. LCMS indicated that the starting material was consumed and the solution was carried out directly for assay analysis. MS (m/z) 276.1 (M⁺−1).

### Example 3

### Neuraminidase Inhibition Assay

A standard fluorimetric assay was used to measure influenza virus neuraminidase (NA) activity. The assay measures the fluorescent product, 4-methylumbelliferone, released from the fluorogenic substrate 4-methylumbelliferyl-N-acetyl-neuraminic acid (MUN) by the enzymatic activity of influenza viral neuraminidase. Two influenza A virus strains were used in the assay: A/H1N1/WSN/33 and H3N2/A/Udorn/72.

The NA activity for each virus was determined before it was used in the inhibition assay. The titration of NA activity was performed through serial dilutions of each virus isolate in the absence of drug. The dilution of virus giving fluorescence counts in the exponential phase (around 100,000) was used to determine the concentration of inhibitor required to inhibit NA activity by 50% (IC₅₀) by incubating serial dilutions of the inhibitor with a constant amount of virus.

To assess the inhibition of neuraminidase by different compounds, the assay mixture contained inhibitor at various concentrations and virus suspension (A/H1N1/WSN/33 or H3N2/A/Udorn/72) in 32.5 mM MES [2-(N-morpholino)-ethanesulfonic acid] buffer—4 mM calcium chloride (pH 6.5) and was incubated at 37° C. on a shaker for 30 minutes. The reaction was started by the addition of the substrate. After incubation with shaking for 60 minutes at 37° C. to allow interaction of drug and virus, the reaction was terminated by adding 0.014M NaOH in 83% ethanol. The fluorescence of the released 4-methylumbelliferone was recorded (excitation at 360 nm and emission at 450 nm), and substrate blanks were subtracted from the sample readings. The 50% inhibitory concentration (IC₅₀) was calculated by plotting percent of fluorescence inhibition of neuraminidase activity versus the inhibitor concentration. The IC₅₀ values were obtained from the graphs by extrapolation, and are the result of 3 independent experiments.

TABLE 1

| | | | | R₄ amino acid | | % inhibition at 500 μM or IC₅₀ (μM) | |
|---|---|---|---|---|---|---|---|
| Compound | R₁ | R₂ | R₃ | R group | R₅ | A/WSN/33 H1N1 | A/Udorn/72 H3N2 |
| DNP-Iso | NH | NO₂ | NO₂ | isoleucine | H | <700 μM | >25% |
| DNP-Iso† | NH | NO₂ | NO₂ | isoleucine | H | ≧40% | |
| NCP-Iso | NH | CN | NO₂ | isoleucine | H | ≧40% | >25% |
| DNP-Val | NH | NO₂ | NO₂ | valine | H | <700 μM | >25% |
| NCP-Val | NH | CN | NO₂ | valine | H | >25% | >25% |
| DNP-Ala | NH | NO₂ | NO₂ | alanine | H | ≧40% | >25% |
| NCP-Ala | NH | CN | NO₂ | alanine | H | >25% | >25% |
| DNP-Gln | NH | NO₂ | NO₂ | glutamine | H | >25% | >25% |
| DNP-Pro* | | | | proline | H | <700 μM | ≧50% |
| DNP-Met | NH | NO₂ | NO₂ | methionine | H | ≧40% | >25% |
| DNP-Ser | NH | NO₂ | NO₂ | serine | H | <700 μM | ≧50% |
| DNP-Arg | NH | NO₂ | NO₂ | arginine | H | >25% | >25% |
| DNP-Gly | NH | NO₂ | NO₂ | glycine | H | >25% | >25% |
| DNP-His* | | | | histidine | | <700 μM | ≧50% |
| DNP-Trp | NH | NO₂ | NO₂ | tryptophan | H | ≧40% | ≧50% |
| DNP-Tyr* | | | | tyrosine | | >1 mM | 5% |

*see above for structure.
†fluorinated in the 5-position of the phenyl ring.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A compound of formula I 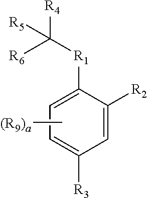
wherein:
R₁ is O, S, NH, or NR₇;
R₂ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NH)NH₂, C(═NR₇)N(R₇)₂, or NR₈;
R₃ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈;
R₄ is an amino acid R group;
R₅ is H or C₁₋₆alkyl;
or R₄ and R₅, taken together, are (CH₂)ₙ, where n is 2 to 6;
R₆ is COOH, sulfonate, phosphonate, or phosphate;
each R₇ is, independently, C₁₋₆alkyl;
each R₈ is, independently, C₁₋₆alkyl or H;
each R₉ is, independently, halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; and
a is 0 to 3;
or a pharmaceutically acceptable salt thereof;
provided that the compound is not dinitrophenyl isoleucine, nitrocyanophenyl isoleucine, dinitrophenyl valine, nitrocyanophenyl valine, dinitrophenyl alanine, nitrocyanophenyl alanine, dinitrophenyl glutamine, dinitrophenyl methionine, N-2,4-dinitrophenyl-DL-methionine sulfone, dinitrophenyl serine, dinitrophenyl arginine, dinitrophenyl glycine, and dinitrophenyl tryptophan.

2-12. (canceled)

13. A compound, or a pharmaceutically acceptable salt thereof, of claim 1 wherein:
R₁ is O, NH, or NR₇;
R₂ is halogen, CN, NO₂, C₁₋₆alkyl, CONHR₇, NHCOR₇, COOR₇, C═NOH, C(═NH)NH₂, C(═NR₇)N(R₇)₂, or NR₈;
R₃ is halogen, CN, NO₂, C₁₋₆alkyl, CONHR₇, NHCOR₇, COOR₇, C═NOH, C(═NH)NH₂, C(═NR₇)N(R₇)₂, or NR₈;
R₄ is an amino acid R group;
R₅ is H or C₁₋₆alkyl;
R₆ is COOH;
each R₇ is, independently, C₁₋₆alkyl;
each R₈ is, independently, C₁₋₆alkyl or H;
a is 0 to 3; and
each R₉ is, independently, halogen, CN, NO₂, or C₁₋₆alkyl.

14-31. (canceled)

32. A compound of formula II 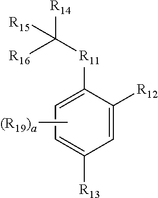
wherein:
R₁₁ is O-phenyl-CH₂—;
R₁₂ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NH)NH₂, C(═NR₁₇)N(R₁₇)₂, or NR₁₈;
R₁₃ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈;
R₁₄ is N(R₂₂)₂;
each R₂₂ is, independently, hydrogen, halogen, C₁₋₆alkyl, or hydroxyl;
R₁₅ is H or C₁₋₆alkyl;
R₁₆ is COOH, sulfonate, phosphonate, or phosphate;
each R₁₇ is, independently, C₁₋₆alkyl;
each R₁₈ is, independently, C₁₋₆alkyl or H;
each R₁₉ is, independently, halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; and
a is 0 to 3;
or a pharmaceutically acceptable salt thereof;
provided that the compound is not dinitrophenyl tyrosine.

33. A compound, or a pharmaceutically acceptable salt thereof, of claim 32 wherein:
R₁₁ is O-phenyl-CH₂—;
R₁₂ is halogen, CN, NO₂, or C₁₋₆alkyl;
R₁₃ is halogen, CN, NO₂, or C₁₋₆alkyl;
R₁₄ is N(R₂₂)₂;
each R₂₂ is, independently, hydrogen or C₁₋₃alkyl;
R₁₅ is H or C₁₋₆alkyl;
R₁₆ is COOH;
each R₁₉ is, independently, halogen, CN, NO₂, or C₁₋₆alkyl; and
a is 0 to 3.

34-36. (canceled)

37. A method of inhibiting neuraminidase in a cell or comprising contacting the cell with an inhibitory effective amount of a compound of formula I, or a pharmaceutically acceptable salt thereof, 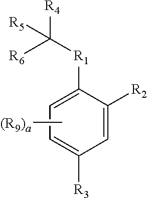
wherein:
R₁ is O, S, NH, or NR₇;
R₂ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NH)NH₂, C(═NR₇)N(R₇)₂, or NR₈;
R₃ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈;
R₄ is an amino acid R group;
R₅ is H or C₁₋₆alkyl;
or R₄ and R₅, taken together, are (CH₂)ₙ, where n is 2 to 6;
R₆ is COOH, sulfonate, phosphonate, or phosphate;
each R₇ is, independently, C₁₋₆alkyl;
each R₈ is, independently, C₁₋₆alkyl or H;
each R₉ is, independently, halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; and
a is 0 to 3.

38-48. (canceled)

49. A method of claim 37 wherein:
R₁ is O, NH, or NR₇;
R₂ is halogen, CN, NO₂, C₁₋₆alkyl, CONHR₇, NHCOR₇, COOR₇, C═NOH, C(═NH)NH₂, C(═NR₇)N(R₇)₂, or NR₈;
R₃ is halogen, CN, NO₂, C₁₋₆alkyl, CONHR₇, NHCOR₇, COOR₇, C═NOH, C(═NH)NH₂, C(═NR₇)N(R₇)₂, or NR₈;
R₄ is an amino acid R group;
R₅ is H or C₁₋₆alkyl;
R₆ is COOH;
each R₇ is, independently, C₁₋₆alkyl;
each R₈ is, independently, C₁₋₆alkyl or H;
a is 0 to 3; and
each R₉ is, independently, halogen, CN, NO₂, or C₁₋₆alkyl.

50-67. (canceled)

68. A method of treating influenza, Pseudomonas aeruginosa, or Bacteroides fragilis infection in a mammal comprising contacting the mammal with a therapeutically effective amount of a compound of formula I, or a pharmaceutically acceptable salt thereof, 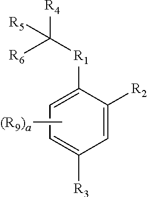
wherein:
R₁ is O, S, NH, or NR₇;
R₂ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NH)NH₂, C(═NR₇)N(R₇)₂, or NR₈;
R₃ is halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈;
R₄ is an amino acid R group;
R₅ is H or C₁₋₆alkyl;
or R₄ and R₅, taken together, are (CH₂)ₙ, where n is 2 to 6;
R₆ is COOH, sulfonate, phosphonate, or phosphate;
each R₇ is, independently, C₁₋₆alkyl;
each R₈ is, independently, C₁₋₆alkyl or H;
each R₉ is, independently, halogen, CN, NO₂, C₁₋₆alkyl, C₁₋₆alkenyl, C₁₋₆alkynyl, C₁₋₆alkoxy, CONHR₇, NHCOR₇, COOR₇, C═NOH, C(═NR₇)N(R₇)₂, or NR₈; and
a is 0 to 3.

69-79. (canceled)

80. A method of claim 68 wherein:
R₁ is O, NH, or NR₇;
R₂ is halogen, CN, NO₂, C₁₋₆alkyl, CONHR₇, NHCOR₇, COOR₇, C═NOH, C(═NH)NH₂, C(═NR₇)N(R₇)₂, or NR₈;
R₃ is halogen, CN, NO₂, C₁₋₆alkyl, CONHR₇, NHCOR₇, COOR₇, C═NOH, C(═NH)NH₂, C(═NR₇)N(R₇)₂, or NR₈;
R₄ is an amino acid R group;
R₅ is H or C₁₋₆alkyl;
R₆ is COOH;
each R₇ is, independently, C₁₋₆alkyl;
each R₈ is, independently, C₁₋₆alkyl or H;
a is 0 to 3; and
each R₉ is, independently, halogen, CN, NO₂, or C₁₋₆alkyl.

81-101. (canceled)

102. A method of treating influenza, Pseudomonas aeruginosa, or Bacteroides fragilis infection in a mammal comprising contacting the mammal with a therapeutically effective amount of a compound of formula II, or dinitrophenyl proline, or dinitrophenyl histidine, or a pharmaceutically acceptable salt thereof, 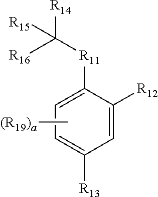
wherein:
R₁₁ is O-phenyl-CH₂—;
R₁₂ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NH)NH₂, C(═NR₁₇)N(R₁₇)₂, or NR₁₈;
R₁₃ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈;
R₁₄ is N(R₂₂)₂;
each R₂₂ is, independently, hydrogen, halogen, C₁₋₆alkyl, or hydroxyl;
R₁₅ is H or C₁₋₆alkyl;
R₁₆ is COOH, sulfonate, phosphonate, or phosphate;
each R₁₇ is, independently, C₁₋₆alkyl;
each R₁₈ is, independently, C₁₋₆alkyl or H;
each R₁₉ is, independently, halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; and
a is 0 to 3;
or a pharmaceutically acceptable salt thereof.

103. A method of claim 102 wherein:
R₁₁ is O-phenyl-CH₂—;
R₁₂ is halogen, CN, NO₂, or C₁₋₆alkyl;
R₁₃ is halogen, CN, NO₂, or C₁₋₆alkyl;
R₁₄ is N(R₂₂)₂;
each R₂₂ is, independently, hydrogen or C₁₋₃alkyl;
R₁₅ is H or C₁₋₆alkyl;
R₁₆ is COOH;
each R₁₉ is, independently, halogen, CN, NO₂, or C₁₋₆alkyl; and
a is 0 to 3;
or a pharmaceutically acceptable salt thereof.

104-113. (canceled)

114. A method of inhibiting neuraminidase in a cell comprising contacting the cell with an inhibitory effective amount of a compound of formula II, or dinitrophenyl proline, or dinitrophenyl histidine, or a pharmaceutically acceptable salt thereof, 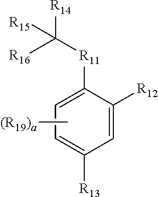
wherein:
R₁₁ is O-phenyl-CH₂—;
R₁₂ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NH)NH₂, C(═NR₁₇)N(R₁₇)₂, or NR₁₈;
R₁₃ is halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈;
R₁₄ is N(R₂₂)₂;
each R₂₂ is, independently, hydrogen, halogen, C₁₋₆alkyl, or hydroxyl;
R₁₅ is H or C₁₋₆alkyl;
R₁₆ is COOH, sulfonate, phosphonate, or phosphate;
each R₁₇ is, independently, C₁₋₆alkyl;
each R₁₈ is, independently, C₁₋₆alkyl or H;
each R₁₉ is, independently, halogen, CN, NO₂, C₁₋₆alkyl, C₁₋₆alkenyl, C₁₋₆alkynyl, C₁₋₆alkoxy, CONHR₁₇, NHCOR₁₇, COOR₁₇, C═NOH, C(═NR₁₇)N(R₁₇)₂, or NR₁₈; and
a is 0 to 3;
or a pharmaceutically acceptable salt thereof.

115. A method of claim 114 wherein:
R₁₁ is O-phenyl-CH₂—;
R₁₂ is halogen, CN, NO₂, or C₁₋₆alkyl;
R₁₃ is halogen, CN, NO₂, or C₁₋₆alkyl;
R₁₄ is N(R₂₂)₂;
each R₂₂ is, independently, hydrogen or C₁₋₃alkyl;
R₁₅ is H or C₁₋₆alkyl;
R₁₆ is COOH;
each R₁₉ is, independently, halogen, CN, NO₂, or C₁₋₆alkyl; and
a is 0 to 3;
or a pharmaceutically acceptable salt thereof.

116-125. (canceled)
